(19) United States
(12) Patent Application Publication
(10) Pub. No.: US 2006/0204457 A1
(43) Pub. Date: Sep. 14, 2006

(54) Cosmetic uv-screen compositions and aminobutadiene-based uv-absorbing complexes therefor

(76) Inventors: Yuzo Toda, Goirle (NL); Sebastianus Gerardus Johannes Maria Kluijtmans, Zeist (NL); Jan Bastiaan Bouwstra, Bilthoven (NL)

Correspondence Address: ROGER PITT;KIRKPATRICK & LOCKHART NICHOLSON GRAHAM LLP, 599 LEXINGTON AVENUE, 33RD FLOOR, NEW YORK, NY, 10022-6030, US

(21) Appl. No.: 10/546,590

(22) PCT Filed: Feb. 25, 2004
(86) PCT No.: PCT/NL04/00139

§ 371 Date: Mar. 21, 2006

(30) Foreign Application Priority Data
Feb. 26, 2003 (EP) 03075572.2
Dec. 24, 2003 (EP) 03079191.7

(51) Int. Cl. A61K 8/64

(52) U.S. Cl. 424/59

(57) ABSTRACT

This invention relates to a cosmetic composition for protection against UV radiation and its detrimental effects comprising a cosmetically and dermatologically acceptable carrier and a UV absorbing compound, not being flavonoid, which is covalently linked to a carrier molecule.

### FIELD OF THE INVENTION

This invention relates to novel cosmetic compositions for protection against UV radiation and its detrimental effects. It also relates to UV-absorbing complexes which are particularly useful in such sunscreen compositions.

### BACKGROUND OF THE INVENTION

The detrimental effects of exposing the skin to UV light are manifold and are well documented in the prior art. It has been long recognized that UV-B radiation, with a wavelength of 290 to 315 nm, causes erythema or sunburn. It was not until around 1980 that it was discovered that UV-A radiation, with a wavelength from 315 to 400 nm, causes phototoxic and photochemical reactions.

While about 70% UV-B radiation is blocked by the outer skin or stratum comeum, this is not the case for UV-A radiation, which can subsequently penetrate deep into the living dermis. A well known destructive effect of UV-A is oxidative stress. Superoxide, which is formed by UV-A radiation, can release iron from ferritin, an iron-storage protein located in fibroblasts in the skin Pourzand et al, Proc. Natl. Acad. Sci. USA, June 1999, Vol 96, p. 6751-56). The role of iron in the Fenton or Haber Weis reaction resulting in the production of highly destructive hydroxyl radicals and hydrogen peroxide is well known. Also other metal ions like copper-ions have been reported to catalyze the formation of oxygen radicals. The role of these destructive products in damaging DNA is well known as for example described by Sestili et al (Free Radical Biology & Medicine [US], Jul. 15, 1998, 25, [2] p. 196-200).

The normal biochemical protection by enzymes like superoxide dismutase is not sufficient to effectively stop the reaction induced by UV-A radiation. Hence the necessity to protect the skin from these harmful effects is still mandatory. Other oxidative stress phenomena are damaging of collagen resulting for example in accelerated skin aging and white spots, or damaging of cell walls by lipid peroxidation.

The use of organic UV absorbers for sunscreen applications is widely known.

A disadvantage of organic UV absorbing compounds is their low water solubility. Therefore, they are unsuitable for use in water-based cosmetics, like hydrogels. They require the use of organic solvents, which generally exhibit unwanted side effects. Lipid soluble UV absorbing compounds, with or without the combination with organic solvents are capable of passing the so-called stratum corneum with the risk of entering the bloodstream.

A further disadvantage of UV absorbing compounds is that they are unstable under UV light. UV exposure can cause photochemical reactions that destroy the UV-absorbing compound thereby reducing the protection against UV radiation.

U.S. Pat. No. 4,839,160 describes a cosmetic formulation for protecting the skin against UV radiation comprising a polymer of benzylidenbornanone units having C4-C12 alkoxy chains. This approach does not result in an increase of the hydrophilicity of the complex. The effect on the penetration throught the skin is mentioned, however the increase of the hydrophobicity will increase the mobility through the lipid elements in the stratum corneum instead of the other way around.

The coupling of UV-A and TV-B absorbing compounds to a polyacrylic acid through an oxygen or nitrogen atom, forming substantially insoluble particles is described in WO 0108647, resulting in a molecular complex designed specifically for the uptake of a-polar compounds to adapt the refractive index into a desired direction. However, mixing with oily compounds has the risk of skin penetration in case of smaller MW complexes that could result in undesired immunogenic reactions.

The use of UV-absorbing complexes, consisting of at least one flavonoid coupled to a bio-polymer is described in WO 03004061. These composite materials however have several disadvantages. The described coupling method is using formaldehyde as reagent which is a highly toxic material, causing strong skin irritations. Besides the formaldehyde coupling method will result in a crosslinking of the polypeptide which is difficult to prevent. Also the stability of the complex obtained from coupling of flavonoids via formaldehyde leaves room for improvement, as has become apparent from stability test under xenon radiation conditions (see example 5). Finally the flavonoid UV absorbing compound absorbs visible light, and this absorption gives the material a yellow color which is not wished for sunscreen materials in cosmetic applications.

EP 1172399 discloses a method to couple organic compounds to a polymer which has at least one reactive amine group. In this patent the use of the synthesized materials for sunscreen applications is not mentioned.

U.S. Pat. No. 5,403,944 discloses an organopolysiloxane polymer with UV-absorbing groups. JP 3161742 describes UV absorbers combined with gelatin for use in photographic sensitive material. No cosmetic applications are mentioned.

In spite of these attempts, there remains a need for UV filters which provide sufficient direct protection from sunlight by blocking the radiation while not showing any risk of allergic or immunogenic nature and not showing any penetration through the skin and being fully transparent for the visible light radiation.

### SUMMARY OF THE INVENTION

It is an object of this invention to provide a new UV-absorbing cosmetic composition.

A further object of the invention is to provide a cosmetic composition comprising a UV-absorbing polymeric compound that can be applied in high concentrations with high stability against breakdown under UV light.

It is also an object of this invention to provide a new, highly efficient, UV-absorbing polymeric compound.

It is a further object of the invention to provide a UV-absorbing polymeric compound that can easily be formulated into a sunscreen composition without giving viscosity and/or stability problems.

Another object of the invention is to provide a new cosmetic composition comprising a UV absorbing polymeric compound which is transparent in the visible light region.

Another object of the invention is to provide a cosmetic composition comprising a UV absorbing polymeric compound of which penetration through the skin is prevented, or at least substantially prevented, subsequently reducing the risk of immunogenic or allergic side reactions.

The present invention is based on the surprising insight that all of the objectives could be reached by a cosmetic composition for protection against UV radiation comprising a cosmetically and dermatologically acceptable carrier and a UV absorbing compound, not being flavonoid, which is covalently linked to a polypeptide as a carrier molecule.

The UV absorbing compound is preferably a broad band UV absorbing molecule, or a UV-A absorbing molecule, more preferably, the UV absorbing compound is a cinnamate, a hydroxybenzophenone, a hydroxy phenylbenzotriazole or an amino-butadiene, or a combination thereof.

It was realised that the UV-absorption of the UV-absorbing complex, i.e an UV-absorbing compound linked to a carrier molecule, should be as high as possible in order to provide sufficient protection against UV-A radiation without giving detrimental effects on the formulation of sunscreen compositions related to for example the viscosity or the (emulsion) stability of the composition.

Surprisingly it was found that by coupling aminobutadiene of formula A to a suitable carrier a UV-absorbing complex was obtained with an unexptected high and efficient UV-absorption.

Thus the invention also relates to a UV-absorbing complex, said complex being a carrier molecule to which is covalently linked aminobutadiene represented by the general formula (A)

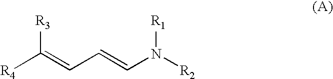

wherein R₁ and R₂, which may be the same or different, each represents a hydrogen atom, an alkyl group having 1-20 carbon atoms, or an aryl group having 6-20 carbon atoms, provided that R₁ and R₂ do not simultaneously represent hydrogen atoms, optionally R₁ and R₂ can combine and form a cyclic amino group;

each of R₁ and R₂ may be substituted by one or more carboxcylic acid moieties; R₃ represents a carboxyl group, —COOR₅, —COR₅ or SO₂R₅ and R₄ represents a carboxyl group, —COOR₆ or —COR₆ wherein R₅ and R₆, which may be the same or different, each represents an alkyl group having 1-20 carbon atoms, or an aryl group having 6 to 20 atoms; optionally R₅ and R₆ can combine and form a 1,3-dioxocyclohexane nucleus, a barbituric acid nucleus, or a 2,4-diazo-1-alkoxy-3,5-dioxocyclohexene nucleus.

Further the invention relates to a method for the preparation of a UV-absorbing complex according to the invention, said method comprising contacting aminobutadiene of formula A with iso-nipecotic acid, or an equivalent thereof, and coupling the resulting product with a carrier molecule.

Also the invention relates to a container comprising such a cosmetic composition and to the use of an UV absorbing compound, not being flavonoid, which is covalently linked to a polypeptide for the preparation of a cosmetic composition for protection against UV radiation.

The new UV absorbing polymer can be applied without significant risk of entering the bloodstream, preventing the risk of immune reactions or other detrimental effects.

### DETAILED DESCRIPTION OF THE INVENTION

“UV absorbing compound” used herein refers to a molecule capable of absorbing UV radiation. If such a UV absorbing compound is linked to a carrier molecule this is refered to as “UV absorbing complex” or “UV absorbing polymeric compound”.

The present invention is directed to UV radiation absorbing compositions that can be used to protect the human skin or hair from the detrimental effects of exposure to sunlight. The present invention is especially directed to cosmetic compositions comprising stable UV absorbing compounds linked to carrier molecules that cannot penetrate through the skin into the bloodstream. Preventing said UV absorbing complex from entering the bloodstream reduces the risk of immune reactions or other detrimental effects.

Surprisingly, a new cosmetic composition comprising a UV radiation absorbing complex comprising at least one UV absorbing compound, not being flavonoid, linked to a polypeptide as a carrier molecule, was found which exhibits all of the desired properties.

The term flavonoid as used herein is meant to be understood as to comprise flavanes, flavanones and flavones and their derivatives.

Within the context of this invention, a polypeptide is to be understood to comprise any molecule having at least 15, preferably at least 20 amino acids linked by peptide bonds, including natural proteins, denatured proteins, synthetic and recombinant proteins and peptides, glycoproteins, proteoglycans, lipoproteins and other molecules which may contain other groups, including bio-oligomer or polypeptide groups as a side chain or in the chain. The maximum length is usually 2000 amino acids. In one embodiment, the polypeptide contains between 15 and 1000, preferably between 30 and 1000 amino acids or between 15 and 500 amino acids. Polypeptides, especially of the gelatin type, having between 30 and 500 amino acids, preferably between 50 and 500 amino acids or between 30 and 300 amino acids are preferred.

In an embodiment the organic UV absorbing compound has no absorption above 400 nm. This means that the organic UV absorbing compound is transparent for visible light.

In another embodiment the organic UV absorbing compound has an absorption in the UV-A region between 315 and 400 nm.

In another embodiment the UV-absorbing complex absorbs less than 10% of its total absorption above 400 nm. This means that the UV-absorbing complex is substantially transparent for visible light. In a preferred embodiment the UV-absorbing complex has 75% or more of its total absorption in the UV-A region between 315 and 400 nm. Thus the invention relates to a cosmetic composition wherein of the UV-absorbing complex less than 10% of the total absorption between 250 and 600 nm is above 400 nm. Also the invention relates to a cosmetic composition wherein of the UV-absorbing complex at least 75% of the total absorption between 250 and 600 nm is between 315 and 400 nm.

In a preferred embodiment the UV absorbing compound is selected from the families of cinnamates (I), hydroxybenzophenones (II), hydroxyphenylbenzotriazoles (III) or amino-butadienes (IV).

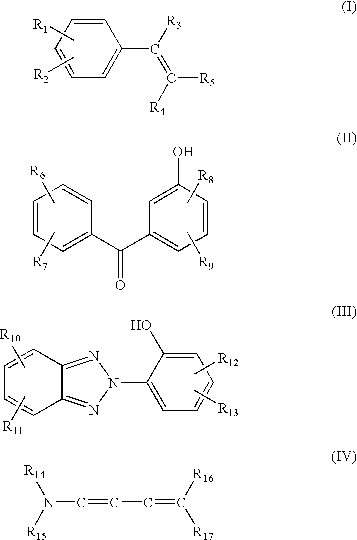

These structures should contain at least one primary amine or carboxylic acid anchor group to attach the UV absorber to respectively the primary amine groups of the lysine amino acid or the carboxy group of glutamic and aspartic acid of gelatin. Examples of coupling strategies are described in EP 0576911, in particular strategies to modify gelatins amine groups with carboxylic acids using carboxylic acid activators.

In a further preferred embodiment the UV absorbing compound is selected from one or more of

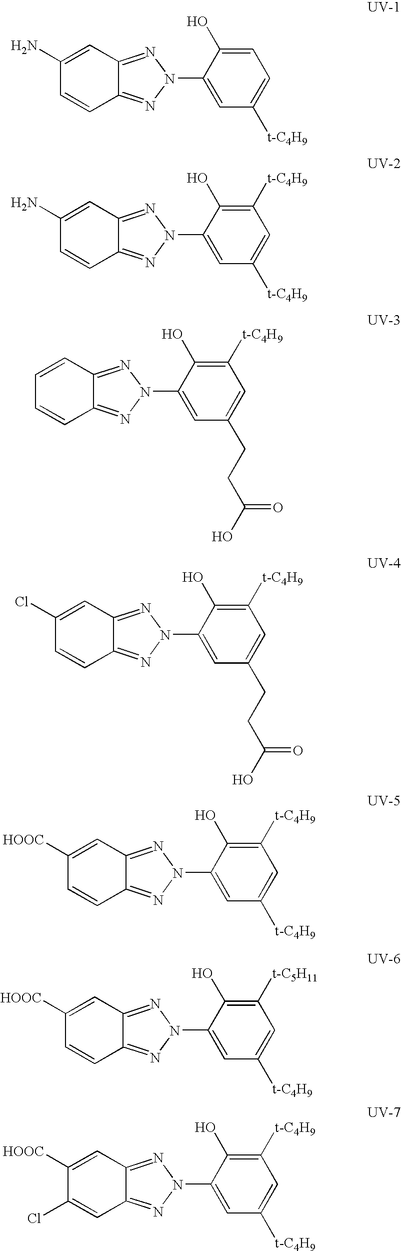

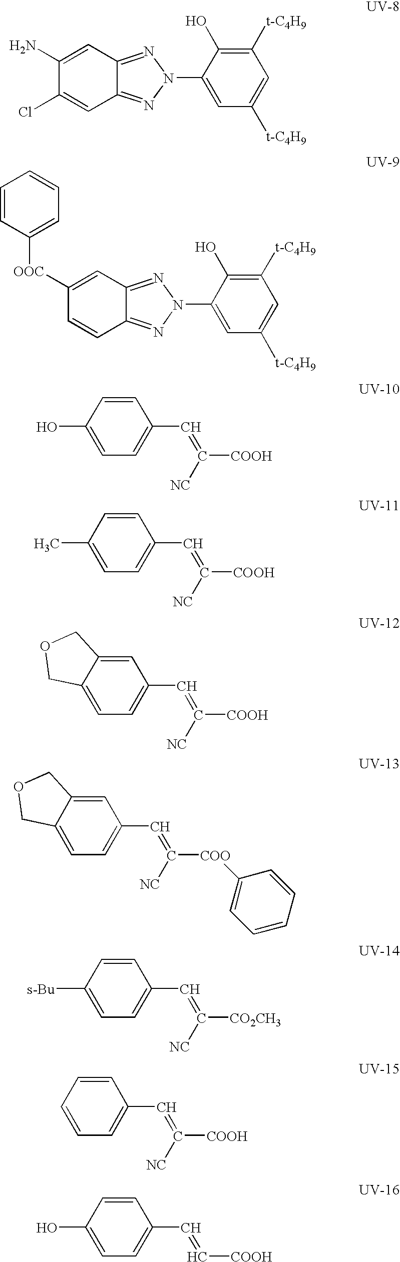

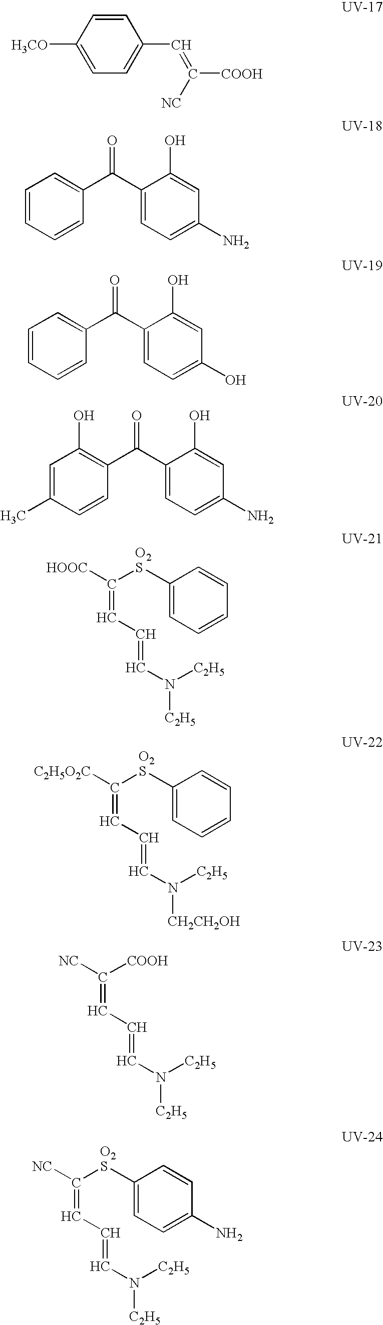

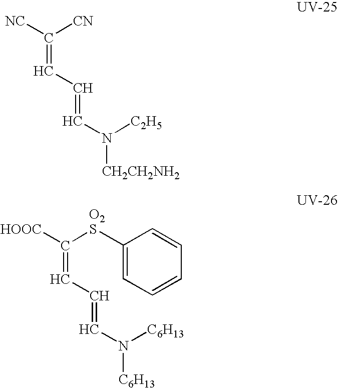

It is preferred that the coupling of the UV absorbing compound is carried out with a method which does not show the risk of polypeptide cross-linking, increasing the UV of the UV absorbing complex in an uncontrolled manner. Especially the reaction of carbodiimide activator with a carboxygroup of the UV absorbing compound followed by conversion of the reactive intermediate to the N-hydroxysuccinimide ester which will react with an amino group in the carrier molecule, in particular bio-polymer or polypeptide, can result in a UV absorbing complex with the desired specifications.

As a method to couple the UV absorbing compounds to the polypeptide, the use of carbodiimide activators followed by conversion of the reactive intermediate to the N-hydroxysuccinimide ester of the UV-absorber is quite convenient. This method is preferable above the one pot reactions described in the WO 03/004061 since the N-hydroxysuccinimide ester may be easily isolated and purified, and the risk of gelatine cross-linking is avoided, see the reaction scheme below.

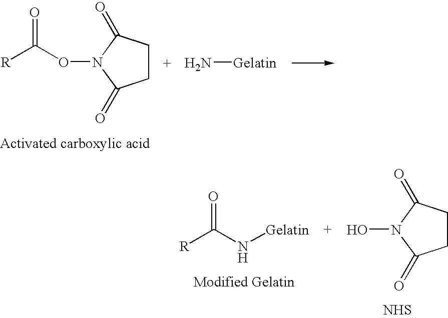

In one embodiment the amount of UV absorbing compound coupled to the carrier molecule is between 3% and 30% by weight.

In another embodiment the polypeptide is a gelatin-like material, this gelatin-like material could be a fish gelatin in case the gelling function of the polymer is not desired and a larger polypeptide is wished.

In one embodiment the polypeptide is a hydrolysed gelatin, having a MW less than 50 kD and more than 1.5 kD, which is adequate to prevent the skin penetration of the UV absorbing polypeptide complex and showing rather limited or even fully absent gelling properties. Preferably the polypeptide has a molecular weight between 3 kD and 30 kD.

The number of UV-absorbing molecules that should be linked to the polypeptide is determined by several factors. The load can be between 0.15 and 15 UV absorbing molecules per 100 amino acids. At lower loads the amount of polypeptide, formulated in a composition in the form of a cream or (hydro)gel or the like, which is put on the skin to achieve the required UV protection could be too high. The number of free reactive groups like amine or carboxyl groups available on the polymer mainly determines the upper limit. For example, a preferred polypeptide, gelatin, can accommodate a load of about 5 UV absorbing molecules per 100 amino acids. Gelatin can be modified to increase the number of available free reactive groups. Too high loads may result in formulation problems. In an embodiment the load may be about between 1 and 10 UV absorbing molecules per 100 amino acids.

By coupling aminobutadiene A to a suitable carrier molecule it was found that the absorption of the aminobutadiene A was unexpectedly high. It was found the carrier molecule could be loaded with a sufficient amount to achieve an UV-absorption of the UV-absorbing complex of at least 5.6 a.u/g.L (=absorption units of an aqueous solution of UV-absorbing complex of 1 gram per liter) at 375 nm. This minimum UV-absorption is preferred for proper functioning of the UV-absorbing complex in sunscreen compositions as it avoids any detrimental effects on the stability and viscosity of the sunscreen formulation. Thus in one embodiment the UV-absorbing complex of this invention has a UV-absorption of at least 5.6 a.u./g.L at 375 nm. Preferably the UV-absorbing complex of this invention has a UV-absorption of at least 20 a.u./g.L, more preferably of al least 25 a.u./g.L, even more preferably of at least 30 a.u./g.L and most preferably of at least 40 a.u./g.L.

The general structure (A) of the aminobutadiene in the UV-absorbing complex of the invention is considered to encompass any equivalent of aminobutadiene (A) which refers to a molecule as represented in formula A that may be modified or substituted but has substantially the same basic structure and still has the property of retaining the high absorption when coupled to a suitable carrier molecule. Such equivalents can be easily imagined by the skilled person. Examples of equivalents of aminobutadiene (A) are found, but not limited to those mentioned, in U.S. Pat. No. 4,195,999.

Of the compounds of the formula (A), those represented by the following general formula (B) are particularly preferred

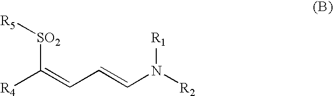

wherein R₁, R₂, R₄ and R₅ have the same meaning as in general formula (A)

In one embodiment the aminobutadiene structure has the following structure formula

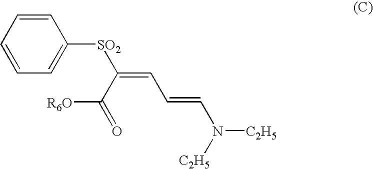

wherein R6 has the same meaning as in general formula A. In a preferred embodiment R₆ represents ethyl. Hereinafter the compound C wherein R₆ is ethyl is referred to as UV-C1.

The aminobutadiene represented above by structure formulas (A), (B), (C) or (UV-C1) may be coupled to a suitable carrier molecule via any carboxylic acid group in the aminobutadiene using methods well known to one of ordinary skill. If the aminobutadiene does not contain a carboxylic acid group, such a group can be introduced by methods known by persons skilled in the art. In a specific embodiment aminobutadiene (A) is coupled via a modification of the amino group (NR₁R₂) which introduces an additional carboxylic acid functionality in this group. In particular the aminogroup is substituted by iso-nipecotic acid. It appears this substitution allows additional efficient coupling to carrier molecules and results in unexpected high absorption. Thus upon treatment of aminobutadiene A, B, C and in a preferred embodiment of aminobutadiene UV-C1, with base and contacting the resulting product with iso-nipecotic acid (also known as 4-piperidinecarboxylic acid or hexahydroisonicotinic acid) an additional carboxylic acid functionality is introduced, which may be coupled via methods involving coupling via carboxylic acid functionalities known per se to a suitable carrier. A preferred method of coupling is via the activated ester, preferably the NHS ester, to amine functionalities on the carrier.

Instead of iso-nipecotic acid also an equivalent thereof may be used, as long as an additional carboxylic acid group is introduced in the aminobutadiene molecule. Such equivalents are exemplified by the general formula (D)

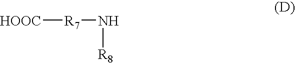

wherein R₇ and R₈, which may be the same or different, each represent an alkyl group having 1-10 carbon atoms; R₈ can also represent a hydrogen atom; optionally R₇ and R₈ can combine and form a cyclic amino group. Preferred equivalents are those which leave the tertiary amino character of the amino group in aminobutadiene (A) intact.

The carrier molecule used to link aminobutadiene (A) to may be any carrier molecule, in particular any polymer, that is capable of coupling aminobutadiene (A) or equivalent thereof. Preferably the amount of aminobutadiene (A) or equivalent thereof that is coupled results in a complex having an UV-absorption of at least 5.6 a.u/g.L. Preferably carrier molecules that are suitable for and allowed in cosmetic compositions, in particular sunscreen compositions are used. Such carrier molecules are known to the skilled person.

In one embodiment the carrier molecule is a polymeric compound, preferably a polymeric compound having free amine groups to couple to the aminobutadiene A.

In another embodiment the carrier molecule is a polypeptide

In a preferred embodiment aminobutadiene A is coupled to said carrier molecule in an amount of at least 7.6 mmol per 100 gram carrier, preferably in an amount of at least 15 mmol per 100 gram carrier.

In another preferred embodiment the amount of aminobutadiene A that is coupled to the polypeptide is between 3% and 50% by weight.

Gelatins which can be used as the water-soluble polypeptide are acid or limne treated skin or bone gelatins from mammals or cold-blooded animals. Such gelatins are common in the prior art and are well described in literature.

Also many chemically modified gelatins are known from the prior art like for example trimellitated, succinylated, acylated, alkylated, phthalated gelatin. Modified gelatins can advantageously be used as long as they do not elicit detrimental reactions like immune reactions when applied to the skin. Succinylated gelatins for example are applied as blood plasma expanders, and are medically and immunologically acceptable gelatin. Alkylated gelatines, see e.g. DE 19721238 for alkyl succinated gelatin, can be used to alter the interaction of the polypeptide with the skin and hence influence the water fastness of the UV absorbing polymer. Thus to improve the interaction of the UV-absorbing complexes of the invention with the skin, alkyl groups, such as for example succinyl groups, may additionally be coupled to the carrier in the UV-absorbing complex. Alternatives for succinyl groups are readily known to the skilled person or can easily be identified in terms of having an improved water fastness. Thus in an embodiment the invention relates to UV-absorbing complexes asdefined above wherein the carrier molecule further comprises alkyl groups, preferably succinyl groups, to improve water fastness.

Modified gelatins in which the amount of free amine-groups has been increased can be used to increase the load of UV absorbing compound on the gelatin. EP 0487686 describes a method to convert carboxyl groups into amines. Spacers can be used to increase the amount of free reactive groups of a polypeptide like gelatin. A spacer is a molecule which can be covalently linked to reactive groups of the polypeptide, like carboxyl groups or amine groups of gelatins, said spacer molecule containing at least two reactive groups like amine-groups or carboxyl groups, thus increasing the amount of available active groups. Spacer molecules are exemplified by amino acids like lysine, glutamic acid or aspartic acid, but are not limited to such structures. For example, activated esters of N-hydroxy-succinimide (NHS) can be used to form peptide linkages between functional groups, like carboxyl groups and primary amines, as is well known (Anderson et al., 1964, J. Am. Chem. Soc. 86:1839-1842, U.S. Pat. No. 5,366,958). NHS can be activated by forming an ester bond between NHS and the functional group (e.g. dihydroxybenzoic acid or an analogue thereof) which should be superimposed onto those of gelatin. Coupling of an activated NHS ester to amino groups of gelatin yields a chemically modified gelatin while N-hydroxy-succinimide is liberated. As another example, the functional group can be coupled to lysine, and the carboxyl group of the modified lysine can then be activated with NHS and coupled to gelatin; alternatively, lysine can be bound to gelatin as a branch, and its two amino groups can be used to bind UV absorbing molecules, not being flavonoid molecules.

Other polypeptides with free amine groups, like casein, sericin, soluble collagen, and the like, can be used, although there is a preference for gelatin or collagen because of their low antigenicity and immunogenicity. If there is a desire to avoid immune reactions even further, for example in case of individuals suffering from (auto-)immune diseases, gelatins incapable of helix-formation can be advantageously used. This can be achieved by chemically modifying gelatins to impair helix formation or, preferably, by using gelatins produced by recombinant techniques as described in EP 1063665. Such recombinant gelatins may lack prolines in the peptide backbone or hydroxylation to hydroxy-proline can be reduced or prevented or avoided. Such recombinant gelatins are preferred over modified gelatins because they are less likely to elicit immune reactions.

A further advantage of gelatins having no or little hydroxy-proline is that such gelatins have a low gelling temperature or do not gelate at all. Less or no gelation makes it possible to use a wider concentration range of the UV-absorbing complex in which the carrier molecule is a gelatin polymer in cosmetic applications. In that respect also gelatin derived from creatures living in cold water can be advantageously used since their gelatin has a naturally low hydroxy-proline content, and thus a low gelling temperature and immunogenicity or antigenicity.

The depth of penetration in the skin can be advantageously regulated by controlling the molecular weight of the gelatin preventing it from entering the bloodstream. Prior art applications of UV absorbing compounds do not include a method to control the skin penetration, but allow skin penetration freely. In this invention penetration of the skin is prevented.

High molecular weight of gelatins, over 200 kD, is less preferred. The molecular weight of conventional natural or modified gelatins can be regulated by for example hydrolysis or by enzymatic breakdown of the gelatin. The desired average molecular weight can be regulated satisfactorily, however the width of the molecular weight distribution can be problematic. After breakdown of the gelatin too small polypeptide (<=3 kD) or too big protein chains can be removed by known techniques like ultrafiltration, dialysis, noodle washing and the like. Recombinantly produced gelatins have the additional advantage that the molecular weight and the distribution can be regulated precisely. Another method to regulate skin penetration is to adjust the hydrophobicity of the protein or polypeptide by chemical modification of said protein or polypeptide, covalently linking hydrophilic or hydrophobic groups to said protein or polypeptide. Thus in one embodiment of this invention the polypeptide is collagen or gelatin which is produced by recombinant DNA technologies.

Advantageously it was found that the UV-absorbing compounds are more stable when coupled to a carrier molecule. When exposed to light the rate of degradation or decomposition of the UV-absorbing compounds is reduced.

The UV-absorbing complex can be advantageously used for the preparation of cosmetic or sunscreen compositions to protect the skin or hair from UV radiation.

Various forms of cosmetic compositions for skin protection comprising UV-absorbing compounds are available in the market such as lotions, emulsions, creams, milks, gels and the like. These may contain oil and/or alcohol. Also aerosols or sticks are known to be used. All such forms of cosmetic compositions may function as a medium to apply the UV-absorbing complex of the invention.

One skilled in the art will be able to select suitable cosmetically and dermatologically carriers that can be used in the sunscreen composition of the invention, in particular in combination with polypeptide to which the UV absorbing compounds, more particularly aminobutadiene A is linked.

The sunscreen composition may contain a second, conventional, UV-absorbing compound. This can be a UV-A, UV-B or broadband UV absorbing compound as described in for example EP 1055412. Other additives as applied in the art can may also be used.

It will be clear to a person skilled in the art that the advantage of linking compounds to a carrier molecule, in particular a polypeptide like gelatin is not limited to UV-absorbing compounds. For example, vitamins like vitamin C and vitamin E are normally added in cosmetic preparations as anti-oxidants. Although they do not impose a danger to the human organism, their function is reduced when such vitamins are allowed to diffuse away through the skin. Such compounds can also be advantageously linked to for example gelatin.

The cosmetic compositions of the present invention can contain in addition to the UV absorbing polypeptide various adjuvants conventionally present in cosmetic compositions of this type for example hydrating agents, emollients or thickening agents, surfactants, preservatives, perfumes, dyes, etcetra.

In one embodiment the carrier molecule, preferably a polypeptide, is water-soluble. By applying water soluble UV absorbing polymeric compounds, the use of organic solvents can be avoided or minimized. Such solvents, besides eliciting immune reactions like skin hypersensitivity, may increase skin penetration by facilitating transport of harmful substances through the lamellar intercellular lipid barrier of the stratum corneum. Applying the inventive cosmetic composition comprising UV absorbing polypeptide complex makes the use of oils or alcohols obsolete, and even helps to prevent unwanted effects like immune reactions to such substances or reduced barrier functions of the skin. The inventive cosmetic composition comprising UV absorbing polypeptide complex is therefore preferably applied as, but not limited to, oil-less compositions like hydrogels.

In one aspect the invention relates to a sunscreen composition comprising less than 18 wt %, preferably less than 13 wt %, more preferably less than 10 wt % UV absorbing complex according to the invention.

### EXAMPLE 1

### Coupling of UV-3 to Gelatin

First the N-hydroxysuccinimide (NHS) ester of UV-3 is prepared by mixing of 4.07 g UV-3 in 120 ml tetrahydrofuran (THF) with 1.38 g NHS and 2.45 g dicyclohexyl carbodiimide (DCC). After 24 hrs the formed DCU is filtered off, and THF is evaporated. The resulting crude NHS-ester is purified by crystallisation from cyclohexane/toluene, filterered and dried. The final yield is 88%.

Coupling to gelatin is performed in DMSO. 2 g of limed bone gelatin is dissolved in 40 ml DMSO at 50° C. 0.26 g NHS-ester of UV-3 is added. The mixture is stirred for 10 hours and precipitated in cold ethyl acetate. After filtration and washing with ethyl acetate and aceton the UV-gelatin is dried. Absence of gelatin crosslinking and non-coupled UV-3 is confirmed by gel permeation chromatography. The load of UV-3 coupled to gelatin is 0.26 mmol/g gelatine as determined by UV-vis spectrophotometry.

### EXAMPLE 2

### Coupling of UV-10 to Gelatin

The NHS-ester of UV-10 was prepared by mixing 2.26 g UV-10 in 120 ml THF, 1.38 g NHS and 2.45 g DCC. After filtration of the DCU and evaporation of THF the crude NHS ester of UV-3 is obtained. The product is further purified by washing with hot tert-butyl methyl ether. Final yield is 95%.

Coupling to gelatin: 9 g of limed bone gelatin is dissolved in 81 g water at 55° C. The pH is adjusted to 7 with 1 M sodium hydroxide. Then a solution of 0.5 g NHS ester of UV-10 in 10 ml THF is added and the reaction mixture is stirred for 4 hours, while keeping the pH at 7 with 1 M sodium hydroxide. After filtration the UV gelatin is dialysed for 48 hours against water at 40° C. using a 10 kD dialysis membrane. Absence of gelatin crosslinking and non-coupled UV-10 is confirmed by gel permeation chromatography. The load of UV-10 is 0.08 mmol/g gelatine, as measured by UV-Vis spectrophotometry.

### EXAMPLE 3

### Coupling of UV-21 to Gelatin

The NHS-ester of UV-21 is prepared by mixing 3.71 g UV-21 in 120 ml THF, 1.38 g NHS and 2.45 g DCC. After filtration of the DCU and evaporation of THF the crude NHS ester of UV-21 is obtained. The product is further purified by crystallisation in toluene/cyclohexane. The final yield is 76%.

Coupling to gelatin: 9 g of limed bone gelatin is dissolved in 81 g water at 55° C. The pH is adjusted to 7 with 1 M sodium hydroxide. Then 0.73 g of NHS ester of UV-21 is added in 10 ml THF and the reaction mixture is stirred for 4 hours, while keeping the pH at 7 with 1 M sodium hydroxide. After filtration the UV gelatin is dialysed for 48 hours against water at 40° C. using a 10 kD dialysis membrane. Absence of gelatin crosslinking and non-coupled UV-21 is confirmed by gel permeation chromatography. Load of coupled UV-21 is 0.10 mmol/g gelatin, as measured by UV-Vis spectrophotometry.

### EXAMPLE 4

### UV-A protection by a UV-21-Gelatin Complex with a Load of 0.1 Millimol UV-21 per Gram Gelatin

The test is performed on volunteer subjects with normal skin types III (Burns moderately, tans gradually) or IV (Burns minimally; tans well). The subjects have no history of photo-sensitivity, are not using systematic medications and have not been exposed to direct sunlight for the previous 6 weeks.

Two test sites of 5×10 cm, each test site divided in 6 sub-sites, are marked on the back of the subjects between the waist line and the shoulder blades. One test site is not treated and therefor unprotected. On the other test site 100 mg sunscreen product is applied uniformly to the skin so that the amount of the UV-absorbing compound applied is about 1 mg per m².

In case of the UV-21-gelatin compound the amount applied is such that also in this case 1 mg/m² of the linked UV-21 is applied, The applied sunscreen is allowed to dry for about 15 minutes.

A series of six doses of UV-A radiation, increasing in energy is applied to each test site using a UV-A Sellas Sun 2000 (irradiance about 50 milliWatt/cm² at 30 cm). Doses applied are from 33 to 8 joule/cm2, randomly applied over the 6 sub sites, each exposure being 25% less than the previous.

The MPD (minimal pigmenting dose) of each test site is determined visually after 3 hours of exposure. The MPD is defined as the quantity of radiation energy required to produce the first unambiguous pigmented reaction.

The SPF (Sun protection factor) is calculated as the quotient of the MPD of the unprotected skin and the MPD of the protected skin.

The results are summarized in the following table:

| MPD (Joule/cm²) | | |
|---|---|---|
| protected | unprotected | SPF |

Group 1: UV-21

| | | | |
|---|---|---|---|
| Volunteer A | 20 | 10 | 2 |
| Volunteer B | 23 | 11 | 2.1 |
| Group 2: UV-21-gelatin complex | | | |
| Volunteer C | 42 | 12 | 3.5 |
| Volunteer D | 37 | 9 | 4 |

The results show that the TV-21-gelatin complex of the invention provides good protection from UV-A radiation. It performed better than the free UV-21 because of the higher stability under UV exposure of the TV-21 linked to gelatin.

### EXAMPLE 5

### Dye Stability Under Xenon Test Conditions; UV Absorption after Xenon Exposure

Measurements are carried out at the Ci 4000 Xenon Weather-Ometer of Atlas Electric Devices Company. The extinction wavelength spectrum represents sunlight spectrum. Experiments are carried out with samples consisting of thin layers of UV absorbing complexes coated on TAC base materials, and dryed under air before Xenon radiation was started.

Solutions for coating on the TAC sheet materials are prepared by making a 5% gelatin solution of UV-gelatin in a suitable solvent (water/ethanol 50:50) for 30 minutes at a temperature of 40 C.

A fixed concentration of the UV absorbing complex was applied with a concentration of 5*10⁻³ Mol/L

UV absorption of samples is measured with standard UV spectrophotometer system. UV absorption is directly correlated to the number of functional molecules in the samples after the Xenon treatment, and the results are listed in the following table.

UV absorption after Xenon radiation (in hours treatment time).

| | 0 | 40 | 80 | 120 |
|---|---|---|---|---|
| | hours | hours | hours | hours |
| UV-22 (non-coupled) type | 0.90 | 0.53 | 0.28 | 0.17 |
| UV-22-gelatin (0.1 mmol UV22/gr | 0.90 | 0.68 | 0.51 | 0.40 |
| gelatin) inventive | | | | |
| UV-22-gelatin (0.15 mmol UV22/gr | 0.90 | 0.65 | 0.50 | 0.38 |
| gelatin) inventive | | | | |
| UV-3 (non-coupled) type | 0.62 | 0.37 | 0.25 | 0.15 |
| UV-3-gelatin (0.2 mmol UV-3/gram | 0.62 | 0.49 | 0.38 | 0.32 |
| gelatin) inventive | | | | |
| UV-3-gelatin (0.25 mmol UV-3/gram | 0.62 | 0.48 | 0.36 | 0.30 |
| gelatin) inventive | | | | |
| Quercetin type | 0.85 | 0.53 | 0.20 | 0.14 |
| Quercetin-gelatin type | 0.85 | 0.60 | 0.27 | 0.23 |

### EXAMPLE 6

### Penetration Depth of UV-21 Into the Skin

A/Color Reaction

The essential condition is the existence of specific detection method, which can be used to visualize the presence of particular sunscreen component. In case of UV-21 one can take advantage of the existence of a fluorescence function of the molecule under radiation with 350 nm wavelength.

B/Skin Penetration

Skin penetration study were executed using the following preparations:

1. UV-21 (1%) in cremor cetomacrogolis FNA (CMC)

2. UV-21-gelatin (low molecular weight fraction, <5 kD) (10-20%) in CMC.

3. UV-21-gelatin (high molecular weight fraction, >5 kD) (10-20%) in CMC.

4. Catechol (1%) (as a penetration marker) in CMC.

The preparations were applied to the skin (buttocks) of three volunteers. The penetration times were 30 min, 1 hour and 3 hours. After each penetration time a biopsy (4 mm) was taken after local anesthesia. The biopsy material was frozen in liquid nitrogen and kept for the further analysis.

Microscopy:

The biopsy materials were cut in consecutive slices of 8 μm thick. The slices were prepared conform standard approaches and the depth of penetration was measured visually under a fluorescence light microscope and expressed as a percentage penetrated epidermis, as follows:

| Penetration depth | 30 min | 1 hour | 3 hour |
|---|---|---|---|
| 1. UV-21 (1%) | 30% | 50% | 80% |
| 2. UV-21-gelatin (small MW) | 10% | 30% | 50% |
| 3. UV-21-gelatin (large MW) | 5% | 20% | 30% |

### EXAMPLE 7

### Coupling of UV-C1 Via the Carboxylic Acid Modified Diethyl Amino Group to Gelatin

The NHS-ester of UV-C1 is prepared according to the following reaction:

To 20 g of UV-C1 in 100 ml water and 300 ml acetonitril was added 10.2 g potassium hydroxide. After heating to 70° C. the reaction mixture was stirred for 2 hours. After vacuum evaporation of the solvents, 300 ml water was added and subsequently 15.4 g isonipecotic acid. The reaction mixture was heated 6 hours at 80° C. After cooling to room temperature, 1 M HCl was added until pH was 2. Then the mixture was extracted twice with ethyl acetate. The combined organic layers were washed with brine and dried on sodium sulfate. The intermediate was isolated by evaporation of the solvents and subsequent co-evaporation with heptanes.

The NHS ester was prepared from the intermediate as follows. To 12 g of the intermediate dissolved in 120 ml THF 3.51 g of N-hydroxy succinimide was added. To this mixture a solution of 6.28 g dicyclohexyl carbodiimide in 50 ml THF was added drop-wise. After 2 hrs the formed precipitate was filtered off and the mother liquor was evaporated. The crude product was dissolved in ethylacetate and washed with water and brine. After solvent evaporation and drying the NHS-ester was isolated. Optionally the product can be further purified by stirring in hot ethanol.

Coupling to Gelatin

Then the isolated NHS-ester is added to hydrolysed limed-bone gelatin (average molecular weight is 21 kD as determined by gel permeation chromatography) in dimethylsulfoxide (DMSO) and after stirring for 12 hrs at 55° C. the gelatin is isolated by subsequent precipitation in ethylacetate, filtered, washed with acetone and dried. The resulting gelatin has a load of 37 mmol/100 g and an UV-absorption of 28 a.u./g L at 375 nm.

### EXAMPLE 8

### Coupling of UV-C1 Via the Carboxylic Acid Modified Diethyl Amino Group to Gelatin

UV-C1 modified gelatin is prepared according to the procedure described in example 7 with a final load of 4 mmol/100 g gelatin and an UV-absorption of 3 a.u./g L at 375 nm

### EXAMPLE 9

### Coupling of UV-C1 Via the Carboxylic Acid Modified Diethyl Amino Group to Gelatin Via a Lysine Spacer

The NHS ester of UV-C1 is prepared as described above in example 7. Hereafter two NHS-esters of UV-C1 are coupled to lysine according to the following reaction:

To 1.17 g lysine H₂O in 70 ml DMSO was added 7 g NHS-ester of UV-C1 in 20 ml DMSO. The mixture was stirred at ambient temperatures for 10 hours and subsequently for 2 hours at 50° C.; 200 ml of dichloromethane was added and the mixture was washed three times with water. After drying with sodium sulfate and evaporation 6.5 g of the double adduct to lysine was obtained. The free carboxylic acid group of the obtained product was converted to the NHS ester by means of the following reaction.

To 6.5 g of the double UV-C1 adduct to lysine in 120 ml dichloromethane was added 1.2 g N-hydroxy succinimide and 2.1 g EDCI coupled to gelatin. The reaction mixture was stirred for 4 hrs at room temperature. The product was purified and isolated by subsequently washing with water, drying, and solvent evaporation.

Coupling of the NHS ester of the double UV-C1 adduct to lysine to gelatin occurs in the same way as in example 7.

The resulting UV-absorbing gelatin shows a even higher absorption than the sample obtained in example 7 (58 mmol/100 g, 44 a.u./gL at 375 nm).

### EXAMPLE 10

### Penetration Depth of Aminobutadiene-Gelatin Into the Skin

A/Color Reaction

The essential condition is the existence of specific detection method, which can be used to visualise the presence of particular sunscreen component. In case of aminobutadiene UV-C1 one can take advantage of the existence of a fluorescence function of the molecule under radiation with 350 nm wavelength.

B/Skin Penetration

Skin penetration studies were executed using the following preparations:

1. Aminobutadiene UV-C (1%) in cremor cetomacrogolis FNA (CMC)

2. Aminobutadiene (UV-C1)-gelatin (low molecular weight fraction) (10-20%) in CMC.

3. Aminobutadiene (UV-C1)-gelatin (high molecular weight fraction) (10-20%) in CMC.

4. Catechol (1%) (as a penetration marker) in CMC.

The preparations were applied to the skin (buttocks) of three volunteers. The penetration times was 10 hours. After 10 hours a biopsy (4 mm) was taken after local anesthesia. The biopsy material was frozen in liquid nitrogen and kept for the further analysis.

Microscopy:

The biopsy materials were cut in consecutive slices of 8 um thick. The slices were prepared conform standard approaches and the depth of penetration was measured visually under a fluorescence light microscope and expressed as follows:

| | Penetration depth after 10 h |
|---|---|
| 1. Aminobutadiene UV-C1 (1%) | + |
| 2. Aminobutadiene (UV-C1)-gelatin | − |
| (MW < 5 kD) | |
| 3. Aminobutadiene (UV-C1)-gelatin | −− |
| (MW > 5 kD) | |

+ = no penetration;
− = limited penetration;
−− = strong penetration

It is clear from these data that the aminobutadiene coupled to gelatin with a UV of more than 5 kD does not penetrate into the epidermis while the aminobutadiene coupled to gelatin with a molecular weight lower than 5 kD does penetrate to a limited extent. The uncoupled aminobutadiene showed the strongest penetration into the epidermis

### EXAMPLE 11

### Stability and Viscosity of UV-Gelatins Applied in Sunscreen Formulation

The aminobutadiene gelatines obtained in examples 7-9 were applied in an acceptable sunscreen formulation in such concentrations that the absorption at 375 nm reached the same value for each sunscreen formulation. Of each sample the stability was monitored and the viscosity was measured. The stability and viscosity of the samples was compared to a control sunscreen formulation

| | Gelatin | | Viscosity |
|---|---|---|---|
| | concentration | | increase |
| Sunscreen formulation | (m/m %) | Stability | compared to 4. |
| 1. Aminobutadiene-gelatin | 20 | − | Large |
| example 8 load 3 a.u./g.L | | | |
| 2. Aminobutadiene-gelatin | 2.0 | +− | Small |
| example 7 load 28 a.u./g.L | | | |
| 3. Aminobutadiene-gelatin | 1.3 | + | Very small |
| example 9 load 44 a.u./g.L | | | |
| 4. No aminobutadiene-gelatin | 0 | + | n.a. |
| added | | | |

− = unstable;
+− = moderately stable;
+ = stable;
n.a. = not analysed

This example clearly shows that the UV-absorbing complexes of this invention provide enough UV-absorption to provide a stable sunscreen formulation with no detrimental effects on the emulsion viscosity.

1. A cosmetic composition for protection against UV radiation comprising a cosmetically and dermatologically acceptable carrier and a UV absorbing compound, not being flavonoid, which is covalently linked to a polypeptide as a carrier molecule.

2. A cosmetic composition according to claim 1, wherein of said UV absorbing compound linked to a carrier molecule less than 10% of the total absorption between 250 and 600 nm of said UV-absorbing compound is above 400 nm.

3. A cosmetic composition according to claim 1, wherein of said UV absorbing compound linked to a carrier molecule at least 75% of the total absorption between 250 and 600 nm is between 315 and 400 nm.

4. A cosmetic composition according to claim 1, wherein said UV absorbing compound is selected from the families of cinnamates (I), hydroxybenzophenones (II), hydroxyphenylbenzotriazoles (III) and amino-butadienes (IV) 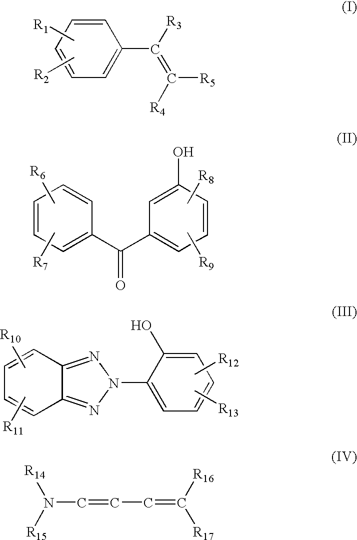
or a combination thereof.

5. A cosmetic composition according to claim 1, wherein said UV absorbing compound is selected from one or more of UV-1, UV-2, UV-3, UV-4, UV-5, UV-6, UV-7, UV-8, UV-9, UV-10, UV-11, UV-12, UV-13, UV-14, UV-15, UV-16, UV-17, UV-18, UV-19, UV-20, UV-21, UV-22, UV-23, UV-24, UV-25 and UV-26.

6. A cosmetic composition according to a of the preceding claim 1, wherein the amount of said UV absorbing compound is between 3% and 50% by weight of said carrier molecule.

7. A cosmetic composition according to claim 1, wherein the number of said UV-absorbing compound molecules linked to the carrier molecule is between 0.15 and 15 UV absorbing compound molecules per 100 amino acids, preferably between between 1 and 10 UV absorbing compound molecules per 100 amino acids.

8. A cosmetic composition according to claim 1, wherein said UV absorbing compound is aminobutadiene represented by the general formula (A) 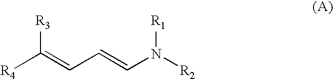
wherein R₁ and R₂, which may be the same or different, each represents a hydrogen atom, an alkyl group having 1-20 carbon atoms, or an aryl group having 6-20 carbon atoms, provided that R₁ and R₂ do not simultaneously represent hydrogen atoms, optionally R₁ and R₂ can combine and form a cyclic amino group;
each of R₁ and R₂ may be substituted by one or more carboxylic acid moieties;
R₃ represents a carboxyl group, —COOR₅, —COR₅ or SO₂R₅ and R₄ represents a carboxyl group, —COOR₆ or —COR₆ wherein R₅ and R₆, which may be the same or different, each represents an alkyl group having 1-20 carbon atoms, or an aryl group having 6 to 20 atoms; optionally R₅ and R₆ can combine and form a 1,3-dioxocyclohexane nucleus, a barbituric acid nucleus, or a 2,4-diazo-1-alkoxy-3,5-dioxocyclohexene nucleus.

9. A cosmetic composition according to claim 1, wherein said UV absorbing compound is represented by the following general formula (B) 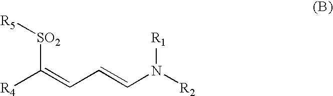
wherein R₁, R₂, R₄ and R₅ are as defined as in claim 1.

10. A cosmetic composition according to claim 8, wherein said carrier molecule is any polymer capable of coupling aminobutadiene (A).

11. A cosmetic composition according to claim 8, wherein said UV absorbing compound linked to a carrier molecule has a UV-absorption of at least 5.6 a.u./g.L at 375 nm.

12. A cosmetic composition according to claim 8, wherein said UV absorbing compound linked to a carrier molecule has a UV-absorption of at least 20 a.u./g.L at 375 nm, preferably at least 25 a.u./g.L, more preferably at least 30 a.u./g.L, most preferably at least 40 a.u./g.L. at 375 nm.

13. A cosmetic composition according to claim 8, wherein at least 7.6 mmol aminobutadiene (A) is coupled to said carrier molecule per 100 gram carrier, preferably at least 15 mmol aminobutadiene is coupled per 100 gram carrier.

14. A cosmetic composition according to claim 1, wherein the carrier molecule further comprises alkyl groups, preferably succinyl groups.

15. A cosmetic composition according to claim 1, wherein said carrier molecule has a molecular weight of between 1,500 and 50,000 Daltons, preferably between 3,000 and 30,000 Daltons.

16. A cosmetic composition according to claim 1, wherein said carrier molecule is selected from casein, sericin, soluble collagen and gelatin.

17. A cosmetic composition according to claim 1, wherein said carrier molecule is collagen or gelatin which is free from helical structure.

18. A cosmetic composition according to claim 1, wherein vitamin C and/or vitamin E is covalently linked to said carrier molecule.

19. A cosmetic composition according to claim 1, wherein said carrier molecule is water-soluble.

20. A cosmetic composition according to claim 1 in the form of a hydrogel.

21. UV-absorbing compound linked to a carrier molecule as defined in claim 4.

22. Method for the preparation of an UV-absorbing compound linked to a carrier molecule according to claim 21 said method comprising contacting aminobutadiene A with iso-nipecotic acid, or an equivalent thereof, and coupling the adduct obtained with said carrier molecule.

23. Use of an UV absorbing compound, not being flavonoid, which is covalently linked to a carrier molecule for the preparation of a cosmetic composition for protection against UV radiation.

24. Sunscreen composition comprising less than 18 wt %, preferably less than 13 wt %, more preferably less than 10 wt % Uw absorbing compound linked to a carrier molecule as defined in claim 1.
